(19) United States
(12) Patent Application Publication
(10) Pub. No.: US 2025/0273324 A1
(43) Pub. Date: Aug. 28, 2025

(54) DEPLOYING MANIFOLD FOUNDATIONAL MACHINE-LEARNING MODEL FOR CLASSIFYING ADDITIONAL DISEASE STATES WITH LIMITED TRAINING DATA

(71) Applicant: Digital Diagnostics Inc., Coralville, IA (US)

(72) Inventors: Michael D. Abramoff, Coralville, IA (US); Abhay Shah, Burlington, IA (US)

(21) Appl. No.: 19/059,131

(22) Filed: Feb. 20, 2025

Related U.S. Application Data
(60) Provisional application No. 63/557,296, filed on Feb. 23, 2024.

(51) Int. Cl. G16H 30/40; G06V 10/70; G06V 10/764; G06V 10/77

(52) U.S. Cl. CPC G16H 30/40; G06V 10/764; G06V 10/7715; G06V 10/87

(57) ABSTRACT

Systems and methods are disclosed herein for classifying one or more disease conditions. In some embodiments, an application stores a common extraction model, the common extraction model trained using training examples for a plurality of diseases. The application stores a plurality of disease classifiers, each disease classifier configured to output whether or not its respective disease is present, each disease classifier trained using training examples for its respective disease. The application receives a selection of a disease and selects a disease classifier from the plurality of disease classifiers corresponding to the disease. The application inputs an image into the common extraction model and receives, as output from the common extraction model, a set of biomarkers extracted from the image. The application inputs the set of biomarkers into the selected disease classifier, the selected disease classifier configured to output whether or not the disease is present in the image.

### CROSS REFERENCE TO RELATED APPLICATIONS

This application claims the benefit of U.S. Provisional Patent Application Ser. No. 63/557,296, filed Feb. 23, 2024, which is incorporated by reference herein in its entirety.

### BACKGROUND

Creating medical artificial intelligence (AI) models for managing, diagnosing and treating requires massive amounts of scarce training data, especially where the AI involves image analysis. Medical training data is scarce for many reasons. For example, deriving data from a human may create harm such as radiation or loss of privacy, and deriving data usually requires substantial resources including staff and expensive sensor equipment available in a limited number of locations. Moreover, clinical data typically has an overabundance of cases, whereas balanced training data, depending on real world prevalence and incidence requires at least as much non-cases (i.e. normal without the specific disease being treated for), which typically do not come to clinic (as there is no need). Yet further, obtaining representativeness of the data in terms of age, sex, race, ethnicity, income and other determinants is practically challenging. High performing models typically require very large amounts of individual examples, varied on a wide range based on the specific application to ensure asymptotic performance saturation and representativeness of the training data. Thus, data scarcity inhibits machine learning and artificial intelligence solutions for managing, diagnosing, and treating disease.

### SUMMARY

In various fields of machine learning (ML), foundational models have been created, using unsupervised learning, essentially using all available data, whatever its modality, quality or representativeness to generalize feature extraction across variables and constraints. Instead of such indiscriminate foundational model learning, the systems and methods disclosed herein leverage the commonality of the appearance of pathologic processes, such as hemorrhages or interruption of the normal tissue boundaries, as so-called biomarkers. The commonality of such pathological processes ensures that across diseases, modalities, and quality, these biomarkers are foundational. In addition, almost all disease processes manifest multiple, statistically semi-independent pathological phenotypes (for example both hemorrhages of various type and tissue disruptions), so that it is worthwhile to derive multiple foundational biomarkers-manifolding the foundational modeling. In particular, where a new model is to be trained to diagnose a disease using images, this conventionally requires a full set of training data sufficient to train a new model. The impracticality of scaling large amounts of clinical training data acts as a barrier to deploying autonomous AI diagnostic tools to diagnose disease, and the systems and methods disclosed herein thus exploit deriving manifolds of disease biomarkers using all available data using unsupervised learning and then creating disease-specific models, modality-specific models, and other models with substantial reduced supervised training data.

In some embodiments, systems and methods are disclosed herein for implementing a manifold foundational model that bifurcates diagnosis into multiple models. An extraction model is trained to take an image as an input and to output an indication of biomarkers within the image. Disease classifiers may be trained to take indications of biomarkers as input, and to output whether the indications of biomarkers are indicative of a disease to which each disease classifier is tuned. As new training data is obtained for a given disease classifier, the new training data may be used to train the common extraction model to identify biomarkers, thus improving the accuracy and robustness of the common extraction model. Moreover, this improves the diagnostic capabilities of the given disease classifier even where only a small set of training examples is available, as the given disease classifier has the benefit of robust biomarker extraction from input images and thus can achieve relatively high accuracy from a small number of examples of biomarkers as mapped to disease condition. By enabling accurate training of disease classifiers using only small amounts of training data, autonomous diagnostic AI can be scaled to detect myriad diseases without the expense of clinical studies for each disease.

In some embodiments, an autonomous diagnosis tool stores a common extraction model, the common extraction model trained using training examples for a plurality of diseases. The autonomous diagnosis tool also stores a plurality of disease classifiers, each disease classifier configured to output whether or not its respective disease is present, each disease classifier trained using training examples for its respective disease. The autonomous diagnosis tool receives a selection of a disease, and selects a disease classifier from the plurality of disease classifiers corresponding to the disease. The autonomous diagnosis tool inputs an image into the common extraction model and receives, as output from the common extraction model, a set of biomarkers extracted from the image. The autonomous diagnosis tool inputs the set of biomarkers into the selected disease classifier, the selected disease classifier configured to output whether or not the disease is present in the image.

### BRIEF DESCRIPTION OF THE DRAWINGS

FIG. 1 is an illustrative block diagram of components used in a system for performing autonomous diagnosis using an autonomous diagnosis tool, in accordance with an embodiment.

FIG. 2 is an illustrative diagram of modules of the autonomous diagnosis tool, in accordance with an embodiment.

FIG. 3 is an illustrative diagram of models used by the autonomous diagnosis tool used to autonomously generate one or more disease diagnoses using biomarkers extracted from an image using a common extraction model, in accordance with an embodiment.

FIG. 4 is an illustrative diagram of a training mechanism for training a common extraction model and disease classifiers used to autonomously generate one or more disease diagnoses using biomarkers, in accordance with an embodiment.

FIG. 5 is an illustrative flowchart of a process for performing autonomous diagnosis using a common extraction model, in accordance with an embodiment.

FIG. 6 is an illustrative flowchart of a process for training a common extraction model and disease classifiers for use in performing autonomous diagnosis, in accordance with an embodiment.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### Overview

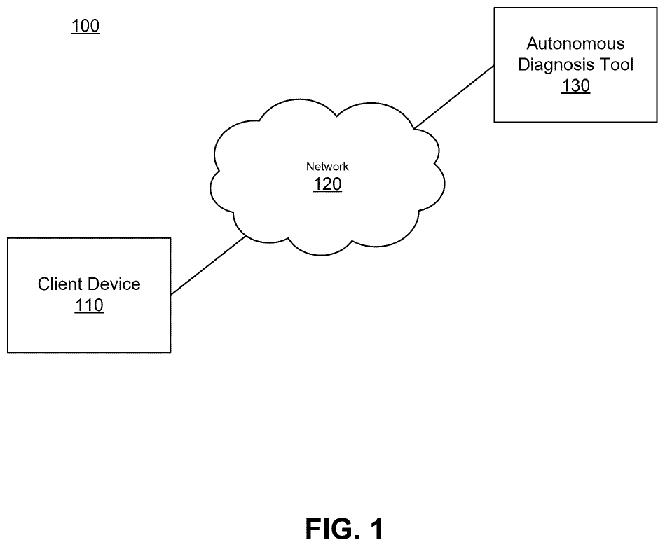

FIG. 1 is an illustrative block diagram of components used in a system for performing autonomous diagnosis using an autonomous diagnosis tool, in accordance with an embodiment. As depicted in FIG. 1, environment 100 includes client device 110, network 120, and autonomous diagnosis tool 130. Client device 110 may be any device or collection of devices used to capture signals from a patient and/or receive input from a physician or other clinical agent involved in processing an autonomous diagnosis of a disease of a patient. An example of client device 110 may be an imaging device used to capture images of a body part of a patient. The images may be of an exterior of the body, of an interior of the body (e.g., ophthalmic imaging such as OCT, x-ray imaging, ultrasound imaging, and the like), or a combination thereof. Other examples of client device 110 may be other sensors that collect patient biometric information and/or repositories of health information about the patient. Client device 110 may also have a user interface for instructing one or more diagnoses be performed based on the signals captured from the patient and for outputting any resulting diagnosis. While only one client device 110 is depicted, any number of client devices 110 may be present (e.g., separate client devices for obtaining images, for processing or transmitting the images, for receiving user input from a patient, a clinician, or another person involved in obtaining an autonomous diagnosis, etc.).

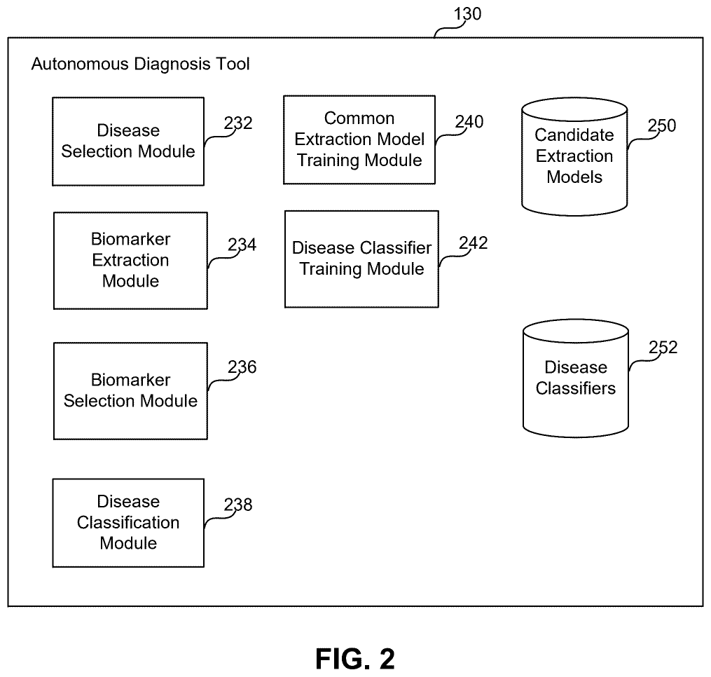

Network 120 may be any network, whether a local network (e.g., Wi-Fi, short-range link, mesh network, etc.) or global network (e.g., the Internet) that enables client device 110 and autonomous diagnosis tool 130 to communicate. Autonomous diagnosis tool 130 may output one or more diagnoses, and may be trained to do so for any additional diseases added over time for diagnosis. Further details of the autonomous diagnosis tool 130 are described with respect to FIGS. 2-6 below.

FIG. 2 is an illustrative diagram of modules of the autonomous diagnosis tool, in accordance with an embodiment. As depicted in FIG. 2, autonomous diagnosis tool 130 includes disease selection module 232, biomarker extraction module 234, biomarker selection module 236, disease classification module 238, common extraction model training module 240, and disease classifier training module 242, as well as candidate extraction models 250 and disease classifiers 252. The module and databases shown in FIG. 2 are merely exemplary, and fewer or more modules and/or databases may be used to achieve the functionality disclosed herein.

Disease selection module 232 selects a disease for which a diagnosis is to be performed. That is, when an image is input into autonomous diagnosis tool (e.g., based on an image captured by client device 110 of a body part of a patient), a diagnosis for one or more diseases may be made based on the image. In some embodiments, out of a plurality of candidate diseases, an administrator may only wish to obtain a diagnosis for a subset of diseases. Disease selection module 232 may receive user input by way of a user interface of one or more diseases, where the image is used to determine whether the patient has those selected diseases.

In an embodiment, disease selection module 232 automatically determines which diseases are to be diagnosed using the image. For example, disease selection module 232 may automatically determine which body part is depicted within the image (e.g., using pattern matching and/or machine learning, where a machine learning model is trained to input the image and to output one or more depicted body parts). Disease selection module 232 may then select one or more diseases associated with the depicted body part(s).

Biomarker extraction module 234 inputs image(s) of a patient (e.g., of one or more body parts of a patient) into a common extraction model. The common extraction model is a machine learning model trained to take an image as an input, and to output one or more biomarkers within the image. Biomarkers are artifacts of interest within an image, and are paired with their location (e.g., location within a given organ or within the image). Exemplary means of extracting biomarkers are discussed in commonly-owned U.S. Pat. No. 11,790,523, entitled “Autonomous Diagnosis of a Disorder in a Patient from Image Analysis,” filed Oct. 30, 2018, issued Oct. 17, 2023, the disclosure of which is hereby incorporated by reference herein in its entirety. Further discussion of biomarker extraction in the context of diagnosis of ear diseases is disclosed in commonly-owned U.S. Pat. No. 11,786,148, entitled “Autonomous Diagnosis of Ear Diseases from Biomarker Data,” filed Aug. 1, 2019, issued Oct. 17, 2023, the disclosure of which is hereby incorporated by reference herein in its entirety. Discussion of obtaining images for autonomous diagnosis is disclosed in the aforementioned patents, and is also discussed in commonly-owned U.S. Pat. No. 11,232,548, entitled “System and Methods for Qualifying Medical Images, filed Mar. 22, 2017, granted Jan. 25, 2022, the disclosure of which is hereby incorporated by reference herein in its entirety.

In some embodiments, biomarker extraction module 234 may perform diagnostics using a vision transformer. The image may be tiled in high resolution, and the tiles may be transformed into embeddings and input into a vision transformer. The vision transformer may directly output a diagnosis and/or may output feature extractions from which a diagnostic model such as the diagnostic model mentioned in the foregoing may determine a diagnosis. Systems and methods of using a vision transformer for these purposes and others are hereby incorporated by reference herein from U.S. patent application Ser. No. 18/955,627, filed Nov. 21, 2024, entitled “High Resolution Medical Image Processing using Vision Transformers and Machine Learning,” the disclosure of which is hereby incorporated by reference herein in its entirety.

The common extraction model is referred to as “common” because, advantageously, it is trained using training data for a plurality of diseases. That is, for example, training data for both diabetic retinopathy and for other diseases of the retina such as macular edema would both be used to train the common extraction model. As training data for new disease classifiers are obtained, that training data can be used to retrain the common extraction model to become ever more robust in detecting biomarkers. To avoid noise, a plurality of candidate extraction modules may exist in candidate extraction models database 250. Biomarker extraction module 234 may select which candidate extraction model to use depending on any heuristic, such as depending on which body part or organ is depicted in an image, where different candidate extraction models may have been trained to detect biomarkers for different body parts or organs. In other embodiments, a single common extraction model may be used for all images.

Disease classifiers take biomarkers as input, and output an autonomous diagnosis for whether the patient has a disease. Using biomarkers as an input, rather than raw images, prevents bias in diagnosis and improves explainability of the diagnosis. For example, where raw images are used to diagnose disease, factors wholly unrelated to the disease might be considered by the machine learning model, such as skin pigment. This can result in racial bias, where the machine learning model may inadvertently be trained to correlate disease with certain skin tones. This also exacerbates explainability constraints, in that it is difficult to determine whether diagnoses are due to detection of disease or other factors (e.g., skin pigment). The aforementioned patent matters that are incorporated by reference herein disclose various means and contexts for using biomarkers as an input and for outputting an autonomous diagnosis.

In some embodiments, biomarker selection module 236 passes all biomarkers extracted by biomarker extraction module 234 to a disease classifier to diagnose a disease. However, this may result in noise where a disease classifier is taking inputs that it is not trained to use in that a disease is not known to correspond to a subset of the biomarkers that were extracted. Therefore, in some embodiments, biomarker selection module 236 selects a subset of extracted biomarkers for use by one or more disease classifiers. Biomarker selection module 236 may determine (e.g., using a mapping table) which biomarkers are associated with diagnosis of each selected disease (as determined by disease selection module 232), and may select those biomarkers as the subset to input into the corresponding disease classifier(s).

Disease classification module 238 inputs the extracted biomarkers (or subset thereof) into each disease classifier of disease classifiers 252 that corresponds to a selected disease. Disease classification module 238 receives, as output from each disease classifier, a determination (e.g., a binary determination, or a probability) as to whether a disease is present in the patient. Where a probability is output, disease classification module 238 may apply a thresholding (e.g., at least 95% likely), and may responsive to determining that a threshold is met or exceeded by the probability, may the disease is present.

In some embodiments, in addition to or instead of outputting a diagnosis based on biomarkers, treatment may be predicted based on the extracted biomarkers. That is, without a need to diagnose a disease, a machine learning model may be trained to predict an intervention that is recommended based on the biomarkers directly. Systems and methods of using machine learning to directly output treatment based on biomarkers are hereby incorporated by reference herein from U.S. patent application Ser. No. 17/541,936, filed Dec. 3, 2021, entitled “Direct Treatment Predictions using Artificial Intelligence,” the disclosure of which is hereby incorporated by reference herein in its entirety. Wherever it is stated herein that a diagnosis may be determined using a machine learning model based on extracted biomarkers, it is equally applicable that a treatment may instead or in addition be output by a trained machine learning model.

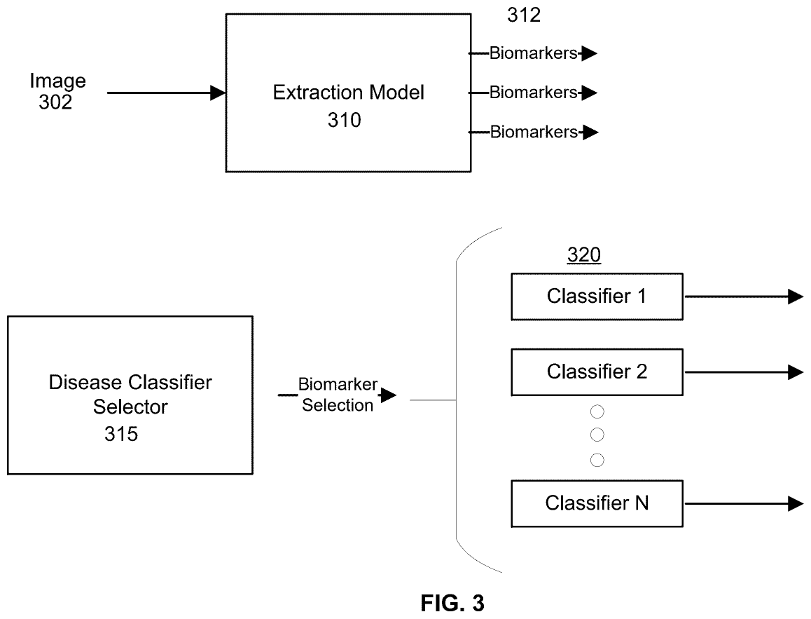

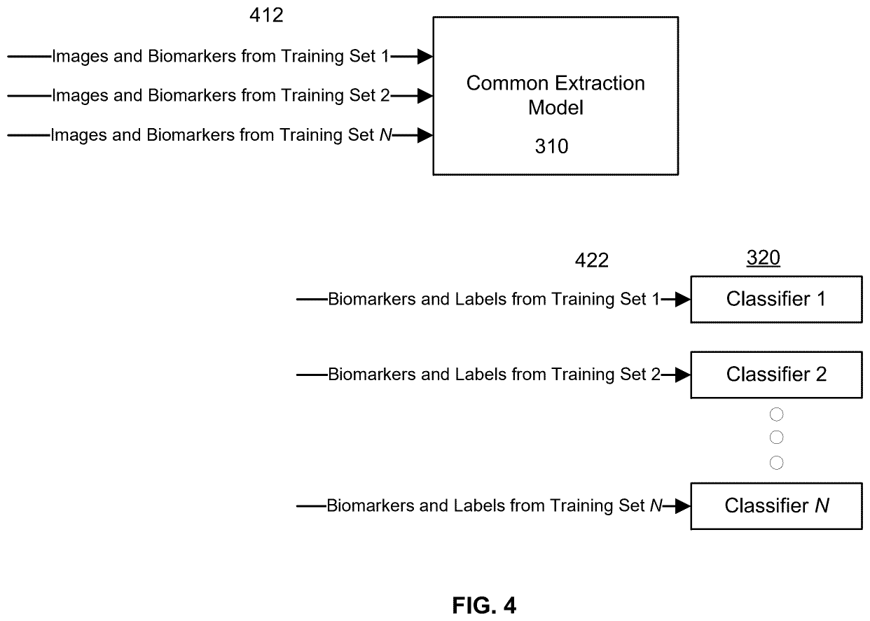

Turning briefly to FIG. 3, FIG. 3 is an illustrative diagram of models used by the autonomous diagnosis tool used to autonomously generate one or more disease diagnoses using biomarkers extracted from an image using a common extraction model, in accordance with an embodiment. As depicted in FIG. 3, image 302 (which may be an image of a patient (e.g., of a patient's body part)) is input into common extraction model 310. Common extraction model 310 outputs biomarkers 312. How common extraction model 310 is trained is described in further detail with reference to FIG. 4 below.

Disease classifier selector 315 may select which disease classifier(s) should be used (e.g., depending on the determinations of disease selection module 232). Optionally, A subset of biomarkers 312 may be selected for input into those classifiers. For the selected disease classifiers 320, the selected biomarkers 312 extracted from image 302 are input, and the classifiers each output a diagnosis as to whether or not image 302 is indicative of the presence of its corresponding disease in the patient.

Turning back briefly to FIG. 2, common extraction model training module 240 is used to train the common extraction model 310, and disease classifier training module 242 is used to train each disease classifier 320. Turning now to FIG. 4, FIG. 4 is an illustrative diagram of a training mechanism for training a common extraction model and disease classifiers used to autonomously generate one or more disease diagnoses using biomarkers, in accordance with an embodiment. Different diseases are associated with their own respective training data. For the purpose of training autonomous diagnosis tool 130, the training data for each given disease includes training examples, where each training example is made up of an image, biomarkers within the image, and a label indicating whether the given disease is present.

Common extraction training model 240 takes, from each disease's training data, the images and biomarkers, and uses those images and biomarkers 412 to train common extraction model 310. With this information, each time a new image is input, common extraction model 310 is able to identify biomarkers within the image. Over time, as new disease classifiers are added to autonomous diagnosis tool 130 with their own respective training examples (e.g., having labels for new diseases), common extraction model 310 is retrained with images and biomarkers from the new training set. This enables common extraction model to become more robust over time in identifying biomarkers that are common to given sets of diseases (e.g., where different common extraction models may exist for different categories of diseases as explained above).

Disease classifier training module 242 similarly takes biomarkers and disease label from each disease's training data, and uses those biomarkers and disease labels to train classifiers 320. Thus, for new images where a given disease is to be classified, biomarkers relevant to that disease can be obtained using common extraction model 310 as input into the classifier corresponding to the given disease, and the classifier is configured to predict whether the disease is present. Classifiers 320 may be expanded over time to include new classifiers for new diseases, where training data may be used to retrain the common extraction model 310.

In this way, even where training data is limited due to a lack of clinical trials, disease classifiers 320 are able to accurately predict diagnoses. This is because the biomarkers that inform the diagnoses are robustly determined using a plethora of additional related training data, and with a robust set of biomarkers produced by the common extraction model, each disease classifier is able to accurately predict a given disease with relatively few examples of biomarkers as mapped to labels. Autonomous diagnosis tool 130 is thereby extensible as a manifold diagnosis model, where any number of diagnoses may be predicted using incrementally added classifiers.

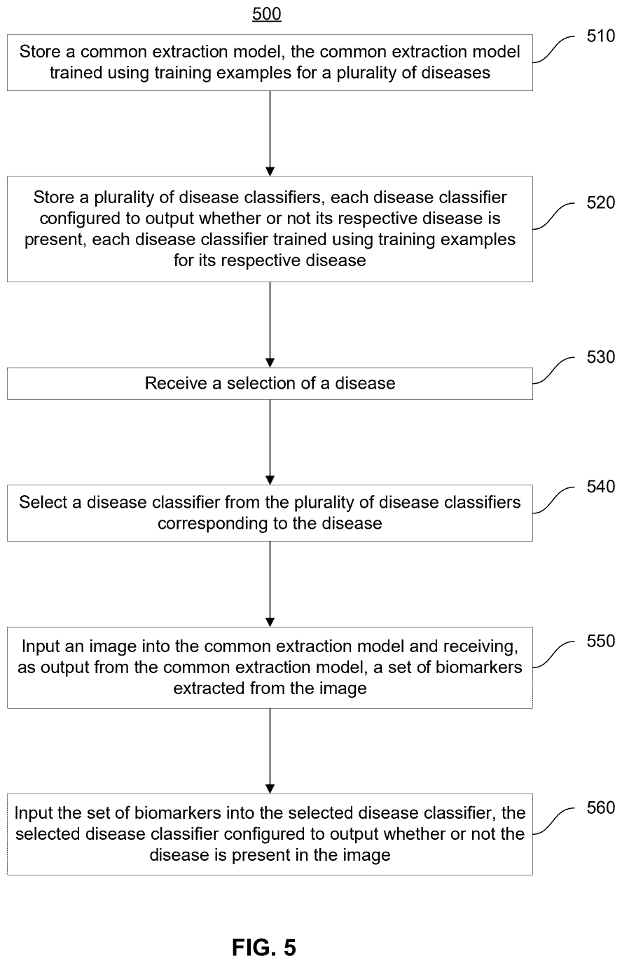

FIG. 5 is an illustrative flowchart of a process for performing autonomous diagnosis using a common extraction model, in accordance with an embodiment. Process 500 begins with autonomous diagnosis tool 130 storing 510 a common extraction model, the common extraction model trained using training examples for a plurality of diseases (e.g., using common extraction model training module 240). Autonomous diagnosis tool 130 also stores 520 a plurality of disease classifiers, each disease classifier configured to output whether or not its respective disease is present, each disease classifier trained using training examples for its respective disease (e.g., using disease classifier training module 242; where treatment may be output rather than disease as mentioned in the foregoing by instead using a treatment classifier).

Autonomous diagnosis tool 130 receives 530 a selection of a disease (e.g., using disease selection module 232), and selects 540 a disease classifier from the plurality of disease classifiers corresponding to the disease. Autonomous diagnosis tool 130 inputs 550 an image into the common extraction model and receives, as output from the common extraction model, a set of biomarkers extracted from the image (e.g., using biomarker extraction module 234). Autonomous diagnosis tool 130 inputs 560 the set of biomarkers into the selected disease classifier, the selected disease classifier configured to output whether or not the disease is present in the image (e.g., using disease classification tool 238).

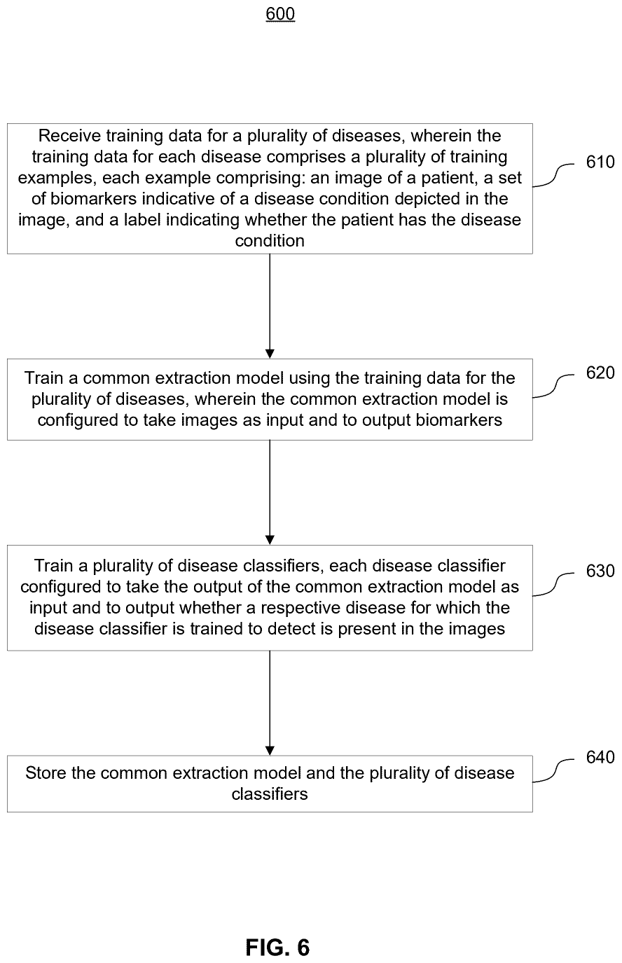

FIG. 6 is an illustrative flowchart of a process for training a common extraction model and disease classifiers for use in performing autonomous diagnosis, in accordance with an embodiment. Process 600 begins with autonomous diagnosis tool 130 receiving 610 training data for a plurality of diseases, where the training data for each disease includes a plurality of training examples. Each example may include an image of a patient, a set of biomarkers indicative of a disease condition depicted in the image, and a label indicating whether the patient has the disease condition. The training data may be for candidate extraction module 250.

Autonomous diagnosis tool 130 trains 620 a common extraction model using the training data for the plurality of diseases, where the common extraction model is configured to take images as input and to output biomarkers (e.g., as common extraction model 310). Autonomous diagnosis tool 130 also trains 630 a plurality of disease classifiers, each disease classifier configured to take the output of the common extraction model as input and to output whether a respective disease for which the disease classifier is trained to detect is present in the images (e.g., as classifier 320). Autonomous diagnosis tool 130 stores 640 the common extraction model and the plurality of disease classifiers.

### SUMMARY

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments of the invention may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. A method for classifying one or more disease conditions, the method comprising:
storing a common extraction model, the common extraction model trained using training examples for a plurality of diseases;
storing a plurality of disease classifiers, each disease classifier configured to output whether or not its respective disease is present, each disease classifier trained using training examples for its respective disease;
receiving a selection of a disease;
selecting a disease classifier from the plurality of disease classifiers corresponding to the disease;
inputting an image into the common extraction model and receiving, as output from the common extraction model, a set of biomarkers extracted from the image; and
inputting the set of biomarkers into the selected disease classifier, the selected disease classifier configured to output whether or not the disease is present in the image.

2. The method of claim 1, wherein the extraction model is one of a plurality of extraction models, each of the plurality of extraction models trained to predict biomarkers for a different group of body parts.

3. The method of claim 2, further comprising selecting the extraction model from the plurality of extraction models based on an identification of a body part depicted in the image.

4. The method of claim 1, wherein the extraction model is retrained responsive to receiving an instruction to train an additional diagnosis model to predict an additional disease.

5. The method of claim 1, wherein the set of biomarkers comprises, for each biomarker within the set of biomarkers, location information as to where the biomarker is located within the image.

6. The method of claim 1, wherein a same group of training examples having an example image, corresponding example biomarkers, and a corresponding disease diagnosis are used to train both the common extraction model and at least one of the plurality of disease classifiers.

7. The method of claim 1, wherein the selection of the disease is performed automatically without user input based on the image.

8. A computer program product for classifying one or more disease conditions, the computer program product comprising a computer-readable storage medium containing computer program code for determining a disease diagnosis that, when executed, causes the computer program product to perform operations comprising:
storing a common extraction model, the common extraction model trained using training examples for a plurality of diseases;
storing a plurality of disease classifiers, each disease classifier configured to output whether or not its respective disease is present, each disease classifier trained using training examples for its respective disease;
receiving a selection of a disease;
selecting a disease classifier from the plurality of disease classifiers corresponding to the disease;
inputting an image into the common extraction model and receiving, as output from the common extraction model, a set of biomarkers extracted from the image; and
inputting the set of biomarkers into the selected disease classifier, the selected disease classifier configured to output whether or not the disease is present in the image.

9. The computer program product of claim 8, wherein the extraction model is one of a plurality of extraction models, each of the plurality of extraction models trained to predict biomarkers for a different group of body parts.

10. The computer program product of claim 9, the operations further comprising selecting the extraction model from the plurality of extraction models based on an identification of a body part depicted in the image.

11. The computer program product of claim 8, wherein the extraction model is retrained responsive to receiving an instruction to train an additional diagnosis model to predict an additional disease.

12. The computer program product of claim 8, wherein the set of biomarkers comprises, for each biomarker within the set of biomarkers, location information as to where the biomarker is located within the image.

13. The computer program product of claim 8, wherein a same group of training examples having an example image, corresponding example biomarkers, and a corresponding disease diagnosis are used to train both the common extraction model and at least one of the plurality of disease classifiers.

14. The computer program product of claim 8, wherein the selection of the disease is performed automatically without user input based on the image.

15. A system comprising:
memory with instructions encoded thereon classifying one or more disease conditions; and
one or more processors that, when executing the instructions, are caused to perform operations comprising:
storing a common extraction model, the common extraction model trained using training examples for a plurality of diseases;
storing a plurality of disease classifiers, each disease classifier configured to output whether or not its respective disease is present, each disease classifier trained using training examples for its respective disease;
receiving a selection of a disease;
selecting a disease classifier from the plurality of disease classifiers corresponding to the disease;
inputting an image into the common extraction model and receiving, as output from the common extraction model, a set of biomarkers extracted from the image; and
inputting the set of biomarkers into the selected disease classifier, the selected disease classifier configured to output whether or not the disease is present in the image.

16. The system of claim 15, wherein the extraction model is one of a plurality of extraction models, each of the plurality of extraction models trained to predict biomarkers for a different group of body parts.

17. The system of claim 16, the operations further comprising selecting the extraction model from the plurality of extraction models based on an identification of a body part depicted in the image.

18. The system of claim 15, wherein the extraction model is retrained responsive to receiving an instruction to train an additional diagnosis model to predict an additional disease.

19. The system of claim 15, wherein the set of biomarkers comprises, for each biomarker within the set of biomarkers, location information as to where the biomarker is located within the image.

20. The system of claim 15, wherein a same group of training examples having an example image, corresponding example biomarkers, and a corresponding disease diagnosis are used to train both the common extraction model and at least one of the plurality of disease classifiers.
